(19) United States
(12) Patent Application Publication
(10) Pub. No.: US 2022/0133534 A1
(43) Pub. Date: May 5, 2022

(54) SMART VITRECTOR

(71) Applicants: Renaud DUVAL, Westmount (CA); Alexandre ABID, Montréal (CA); Flavio REZENDE, Montréal (CA); Christos BOUTOPOULOS, Montréal (CA)

(72) Inventors: Renaud DUVAL, Westmount (CA); Alexandre ABID, Montréal (CA); Flavio REZENDE, Montréal (CA); Christos BOUTOPOULOS, Montréal (CA)

(21) Appl. No.: 17/518,205

(22) Filed: Nov. 3, 2021

Related U.S. Application Data
(60) Provisional application No. 63/109,040, filed on Nov. 3, 2020.

(51) Int. Cl. A61F 9/007; G08C 23/06

(52) U.S. Cl. CPC A61F 9/00736; G08C 23/06

(57) ABSTRACT

A method for controlling a vitrector of a vitrectomy system includes emitting an optical signal over an opening in a body of the vitrector, the opening providing access to a cutting member within the body, and using an optical sensor provided on the vitrector to capture optical feedback produced when the optical signal interacts with material proximate to the opening. The presence of the material in a vicinity of the cutting member of the vitrector is determined based on the optical feedback. A control signal to alter operation of the vitrector is issued responsive to determining the presence of the material in the vicinity of the cutting member of the vitrector.

### CROSS-REFERENCE TO RELATED APPLICATIONS

The present application claims priority on U.S. Patent Application No. 63/109,040 filed Nov. 3, 2020, the entire contents of which are incorporated by reference herein.

### TECHNICAL FIELD

The present disclosure generally relates to the field of medical devices, and more specifically to devices adapted for use in eye surgery and methods of using such devices.

### BACKGROUND

Pars plana vitrectomy (PPV), or simply “vitrectomy”, is a surgical eye procedure which involves removal of the vitreous gel from the eye and subsequent repair of the retina. By allowing controlled access to the posterior segment of the eye, PPV is frequently used to help with the management of diseases of the retina. Common use cases include rhegmatogenous retinal detachments (RRD), diabetic tractional retinal detachments, vitreous hemorrhages, epiretinal membranes, macular holes etc.

When performing PPV, surgeons manipulate surgical instruments inside the eyeball, in close proximity to the retinal layer. In certain cases, this can pose an injury risk to the retinal layer. An instrument used during PPV is a vitrector, which integrates the functions of vitreous gel cutting and aspiration into a single tool. In a typical setup, a surgeon controls the positioning of the vitrector inside the eye with a hand and uses a foot pedal to activate/deactivate the vitrector's cutting and aspiration functions. One step performed during vitrectomy for retinal detachment is the “shaving” of the vitreous base. During this technique, the vitrector is brought into close proximity to the retina, while simultaneously aspirating and cutting the base of the vitreous gel. The closer the vitreous base is shaved to the retina, the more effective the surgery can be. However, this also leads to a higher risk of iatrogenic injury to the retina, which can occur for example if the aspiration and/or cutting functions are not stopped in sufficient time.

When a potentially undesirable situation occurs, such as the retina (rather than the vitreous gel) being aspirated by the vitrector, the surgeon must quickly release the foot pedal to prevent a retinal injury. Human reaction time is limited for physiological and anatomical reasons, and is typically within the range of 300 to 400 milliseconds. Given that the cutting rate of the vitrector is in the range of 1000 to 10,000 cuts per minute, the vitrector can in certain instances perform 5 to 67 cuts before deactivation. Although vitrectors can be set to operate at lower cutting rates, this can produce increased retinal traction, and thus may not be used in close proximity to mobile retina.

Amongst the surgical complications of primary RRD-related procedures, iatrogenic retinal breaks may occur in between 7.7% to 10% of surgeries, and some studies report incidence rates of up to 32.5%. Retinal breaks of this nature are particularly grave because they may lead to failure of the surgery if not identified and appropriately addressed. Given estimates of over 225,000 patients undergoing a vitrectomy in the United States each year, tens of thousands of patients may suffer from iatrogenic retinal breaks on a yearly basis. Decreasing the rate of iatrogenic retinal breaks would improve the success rate of the surgery, reduce the rate of retinal re-detachment and re-operation, and have an overall beneficial effect on both the patients and the health-care system.

While existing vitrectomy techniques are suitable for their purposes, improvements remain desirable.

### SUMMARY

There is accordingly provided a method for controlling a vitrector of a vitrectomy system, the method comprising: emitting an optical signal over an opening in a body of the vitrector, the opening providing access to a cutting member within the body; using an optical sensor provided on the vitrector to capture optical feedback produced when the optical signal interacts with material proximate to the opening; determining the presence of the material in a vicinity of the cutting member of the vitrector based on the optical feedback; and issuing a control signal to alter operation of the vitrector responsive to determining the presence of the material in the vicinity of the cutting member of the vitrector.

The method as defined above and described herein may further include, in whole or in part, and in any combination, one or more of the following additional steps and/or elements.

In certain embodiments, the method further comprising processing the optical feedback and issuing a detection result.

In certain embodiments, the optical feedback comprises light scattering information, and wherein processing the light scattering information comprises generating imaging data by performing optical coherence tomography, and further wherein issuing the detection result comprises providing the imaging data to a detection algorithm.

In certain embodiments, generating the imaging data by performing optical coherence tomography comprises generating at least one amplitude scan based on the light scattering information.

In certain embodiments, the optical signal comprises a laser beam propagating in a first direction and the optical feedback comprises a back-reflection of the laser beam.

In certain embodiments, processing the optical feedback comprises detecting a variation of a power of the laser beam and the back-reflection of the laser beam, and wherein issuing a detection result comprises providing the variation of power to a detection algorithm.

In certain embodiments, the optical sensor is disposed on an outer surface of the body of the vitrector.

In certain embodiments, the optical sensor comprises a fiber-based optical sensor positioned inside a groove formed in an outer surface of the body of the vitrector and extending longitudinally therealong.

In certain embodiments, determining the presence of the material in the vicinity of the cutting member of the vitrector comprises determining whether a retina is approaching the opening of the vitrector.

In certain embodiments, the material is a retina, and further wherein determining the presence of the material in the vicinity of the cutting member of the vitrector further comprises determining a presence of an aqueous/vitreous humour of the patient in the vicinity of the opening of the vitrector.

In certain embodiments, issuing the control signal to alter the operation of the vitrector comprises commanding a stopping of movement of the cutting member of the vitrector.

In certain embodiments, issuing the control signal to alter the operation of the vitrector comprises commanding a reduction in a suction force provided to the vitrector.

There is further provided a vitrectomy system, comprising: a vitrector including a tubular structure defining an opening near a distal end thereof for aspiration of material, and a cutting member located within the tubular structure and translating therein for cutting material which enters the tubular structure via the opening; an optical sensor provided on the vitrector and aligned to emit an optical signal that extends over the opening and to capture optical feedback produced when the optical signal interacts with material proximate to the opening; and a control system coupled to the optical sensor and the vitrector for: receiving the optical feedback from the optical sensor; determining the presence of the material in a vicinity of a cutting member of the vitrector based on the optical feedback; and issuing a control signal to alter the operation of at least one of the cutting member and a suction force source coupled to the vitrector responsive to determining the presence of the material. The vitrectomy system of claim 13, wherein the optical sensor is disposed on an outer surface of the body of the vitrector.

The vitrectomy system as defined above and described herein may further include, in whole or in part, and in any combination, one or more of the following additional elements.

In certain embodiments, the optical sensor comprises a fiber-based optical sensor positioned inside a groove formed in an outer surface of the body of the vitrector, the groove extending longitudinally along the body of the vitrector.

In certain embodiments, the control system coupled to the optical sensor comprises: an optical coherence tomography (OCT) unit communicatively coupled to the optical sensor, the OCT unit configured for receiving optical feedback from the optical sensor, processing the optical feedback, and generating imaging data by performing optical coherence tomography; a detection algorithm communicatively coupled to the OCT unit, the detection algorithm configured to detect the material proximate to the opening of the vitrector and issue a detection signal; and a feedback mechanism communicatively coupled to the detection algorithm and to the vitrector, the feedback mechanism configured to effect a change in the operation of the vitrector in response to the detection signal.

In certain embodiments, the control system further comprises: a broadband light source providing light to the optical sensor; an attenuator provided in a path between the broadband light source and the optical sensor; and a spectrometer communicatively coupled to the optical sensor and to the broadband light source via a coupler, the spectrometer receiving a signal from the optical sensor.

In certain embodiments, the OCT unit comprises the spectrometer, the broadband light source, the attenuator, and the coupler in a common-path configuration.

In certain embodiments, the optical sensor is a fiber-based optical sensor, the vitrectomy system further comprises a reflective element provided on a distal end of the vitrector opposite to the opening, and wherein the control system coupled to the optical sensor further comprises: a laser source configured for emitting a laser beam, the laser beam undergoing reflection on the reflective element; a reference sensor configured for monitoring the power of the laser source; a back-reflection sensor configured for monitoring the power of a back reflection of the laser beam; and laser coupling optics configured for coupling the laser source, the optical sensor, and the back-reflection sensor, the laser coupling optics enabling forward propagation of the laser beam into the fiber-based optical sensor but preventing back-propagation of the laser beam into the laser source; a detection algorithm configured for triggering a shutdown of the vitrectomy system is sufficient power variation of the back reflection of the laser beam is detected.

In certain embodiments, the wavelength of the laser source is selected in the UV, visible, or IR spectra.

In certain embodiments, the fiber-based optical sensor is single mode or multimode fiber.

In certain embodiments, the fiber-based optical sensor includes a focusing design.

In certain embodiments, the laser source is continuous wave or pulsed wave.

In certain embodiments, the vitrectomy system further comprises a vitrectomy machine communicatively coupled to the control system and to the vitrector, the control system issuing commands to the vitrectomy machine for operating the vitrector.

In certain embodiments, the commands comprise instructing the vitrectomy machine to cease providing suction force to the vitrector, to cause the opening of the vitrector to be shut, or to slow or stop the movement of the cutting member.

In accordance with an alternate broad aspect, there is also provided method for controlling a vitrector of a vitrectomy system. Light scattering information is received from an optical sensor associated with the vitrector, the optical sensor disposed on an outer surface of the vitrector. Imaging data is generated by performing optical coherence tomography based on the light scattering information. A medical risk is detected based on the imaging data. A signal is issued to alter operation of the vitrector responsive to detecting the medical risk.

In accordance with an alternate broad aspect, there is provided a vitrectomy system that comprises a vitrector including a tubular structure defining an opening near a distal end thereof for aspiration of material, and a cutting member located within the tubular structure for cutting material which enters the tubular structure via the opening; an optical sensor disposed on an outer surface of the vitrector and aligned to emit an optical signal that extends over the opening; and a control system coupled to the optical sensor and the vitrector. The control system is configured for: receiving light scattering information from the optical sensor; generating imaging data by performing optical coherence tomography based on the light scattering information; detecting a medical risk based on the imaging data; and issuing a signal to alter operation of the vitrector responsive to detecting the medical risk.

Many further features and combinations thereof concerning embodiments described herein will appear to those skilled in the art following a reading of the instant disclosure.

### BRIEF DESCRIPTION OF THE FIGURES

In the figures:

FIG. 1 illustrates a diagram of an exemplary vitrectomy system.

FIGS. 2A-2E illustrate schematic diagrams of embodiments of the vitrectomy system of FIG. 1.

FIG. 3 illustrates a block diagram of an example computing system for implementing part or all of the vitrectomy system of FIG. 1.

FIG. 4 illustrates a flowchart of an example method of controlling a vitrector.

FIG. 5 illustrates an exemplary embodiment of the vitrectomy system of FIG. 1.

FIGS. 6A-6D illustrate an example vitrector testing scenario.

FIG. 7 illustrates example light outputs of an example optical detection system.

FIGS. 8A-8D illustrate an example technique for producing a vitrector.

FIGS. 9A-9B illustrate an example response time testing scenario.

FIGS. 10A-9B illustrate bar graphs of different example outcomes for vitrectomy operations.

### DETAILED DESCRIPTION

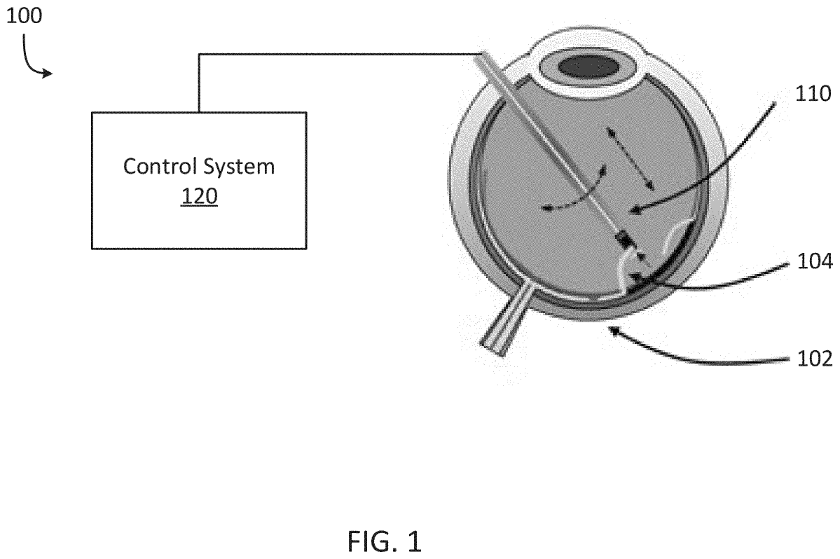

With reference to FIG. 1, a vitrectomy system 100 in accordance with an exemplary embodiment of the present disclosure is illustrated. The vitrectomy system 100 includes generally of a vitrector 110 and a control system 120. The vitrector 110 is communicatively coupled to the control system 120, which controls part or all of the operation of the vitrector 110 in response to various operator input, and using any suitable electrical, electronic, and/or mechanical methods. The vitrectomy system 100 may be operated by a surgeon or other medical professional, as appropriate. The vitrectomy system 100 may also be operated outside of a medical context, for testing purposes or otherwise, by a non-medical professional such as for instance by a technician, engineer, or by service personnel, as appropriate. In a particular embodiment, the vitrectomy system 100 may be used on an eyeball 102 (such as of a human patient, an animal, a cadaver, an artificial implant or simulated component or model), including for instance in surgical ocular procedures relating to conditions of the retina 104 of the eyeball 102. The vitrectomy system 100 may also be used ex-vivo, for instance during equipment testing procedures, to perform inspection, maintenance, calibration and/or repair of the vitrectomy system 100, or for demonstration and/or training purposes. When used in surgical procedures, the vitrector 110 serves to cut the vitreous gel within the eyeball 102 and to aspirate the vitreous gel and/or the aqueous/vitreous humour within the eyeball 102 in order to provide the surgeon with access to the retina 104. By way of some non-limiting additional examples, the vitrector 110 may be used in other procedures relating to the eyeball 102, including to remove so-called “floaters” in the vitreous gel of the eyeball 102, to remove portions of the vitreous gel in an anterior segment of the eyeball 102, as part of performing a pars plana capsulectomy, to remove residual lens fragments within the eyeball 102, and the like.

As will be described in greater detail hereinbelow, the vitrector 110 of the present disclosure includes an optical detection system which is operable to detect the presence of the retina 104 in proximity to the cutting member of the vitrector 110. In some embodiments, the optical detection system includes an optical fiber-based detector which is affixed to an outer body of the vitrector 110 using an adherent substance or other adhesive. It should be noted that in some other embodiments, the optical detection system may be integrated within the vitrector 110. The optical fiber is configured for emitting an optical signal, for instance a beam of light or other emission, based on which the control system 120 may detect the presence of the retina 104 at or near the cutting member of the vitrector 110, and/or the presence of other material at or near the cutting member of the vitrector 110. When the control system 120 detects that damage to the retina 104 may occur, the operation of the vitrector 110 may be modified, for instance to reduce or eliminate the risk of damaging the retina 104.

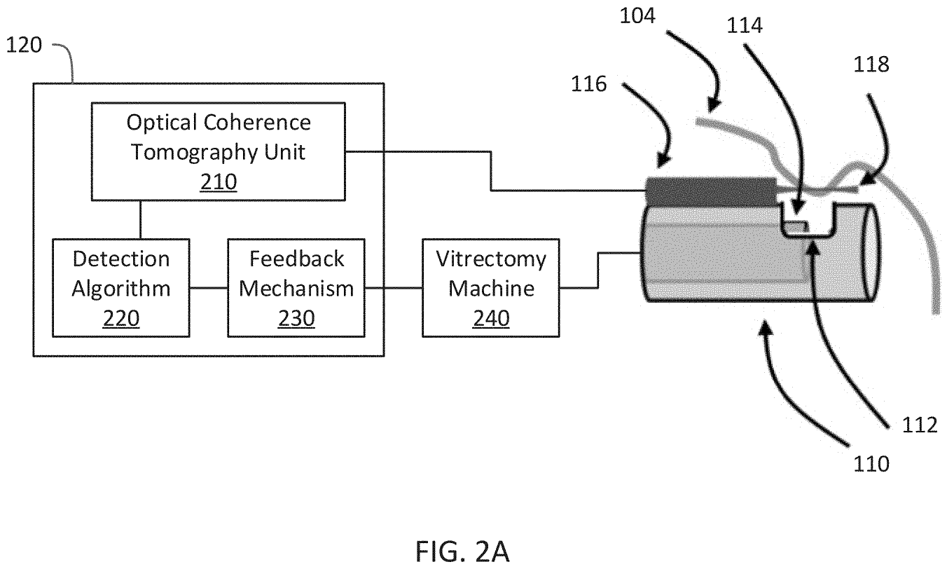

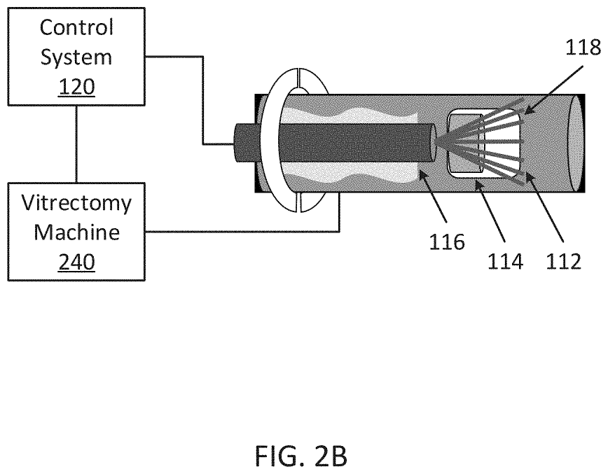

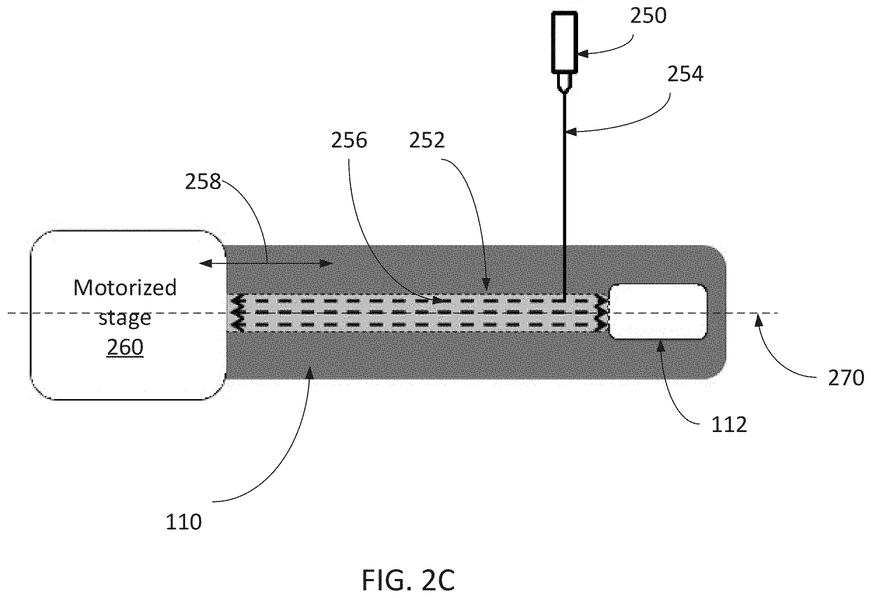

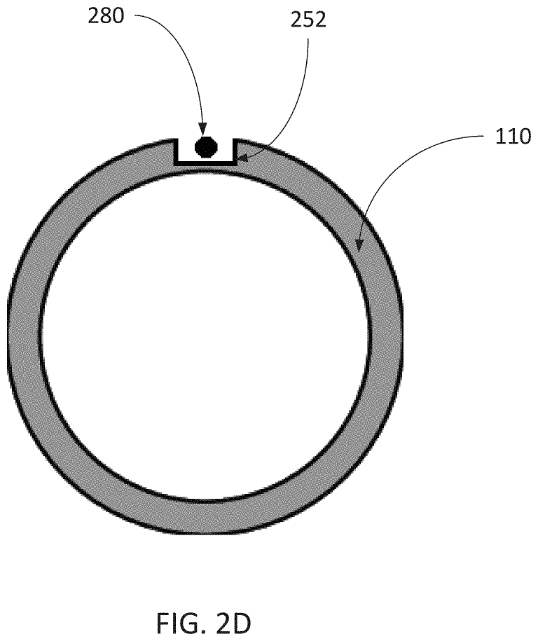

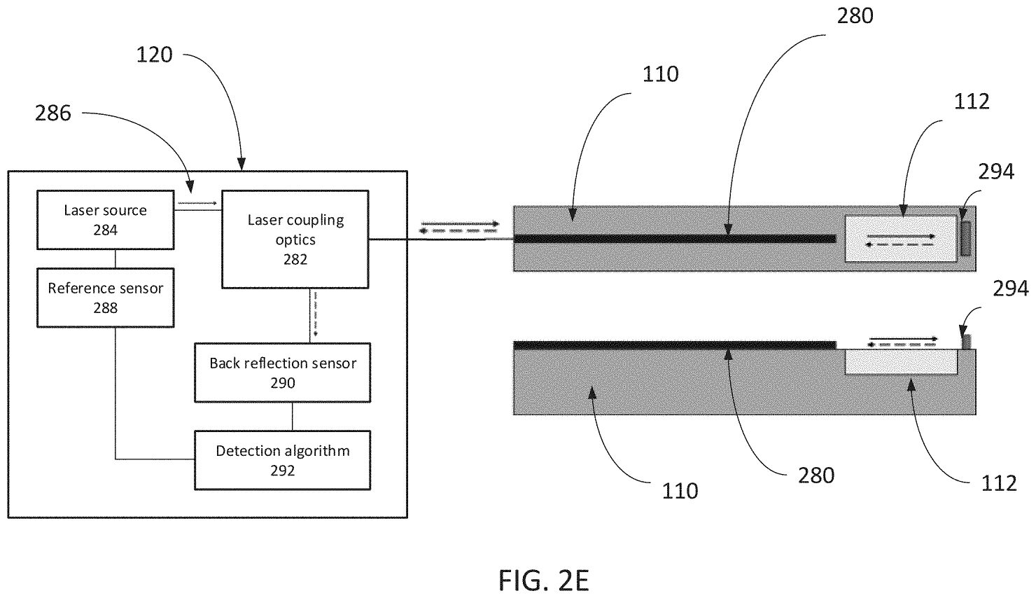

With additional reference to FIG. 2A, example embodiments of the vitrector 110 and the control system 120 are illustrated. The vitrector 110 includes a tubular structure in which is defined an opening 112. Movable within the tubular structure of the vitrector 110, for example in a reciprocating manner, is a cutting member, referred to as a guillotine 114. In operation, the guillotine 114 moves back and forth to cut material which enters through the opening 112. The vitrector 110 also aspirates material via the opening 112, for instance due to a suction force applied to the interior of the tubular structure of the vitrector 110. The vitrector 110 is connected to, and forms part of, a larger vitrectomy system, which also includes a vitrectomy machine 240. The vitrectomy machine 240 can be provided with one or more pumps or similar functionality for applying the suction force used by the vitrector 110 to aspirate material via the opening 112.

The vitrector 110 also includes an optical-sensor 116, which produces an optical signal 118, which may be a light beam. The optical sensor 116 may produce any suitable type of optical signal using any suitable light source. In some embodiments, the optical signal emitted by the optical sensor 116 is a broadband light signal, which is produced by a broadband light source forming part of the optical sensor 116. In some other embodiments, the optical signal emitted by the optical sensor 116 is a coherent light signal, which is produced by a coherent light source forming part of the optical sensor 116, for instance a laser-light source. The light source which forms part of the optical sensor 116 may, in certain embodiments, include the aforementioned optical fiber-based light sources.

The optical sensor 116 is configured for detecting the way in which material near the optical sensor 116 interacts with the optical signal 118, by receiving and/or capturing optical feedback produced when the optical signal 118 interacts with material proximate to the opening. This optical feedback may include reflection or transmission of the optical signal 118, diffraction or refraction of the optical signal 118, optical scattering of the optical signal 118, interference of the optical signal 118, or the like. The optical sensor 116 is affixed to the tubular structure of the vitrector 110, for instance on an outer surface thereof, and is positioned to emit the optical signal 118 so as to be superposed on and substantially parallel to the elongated tubular structure of the vitrector 110. As shown in FIG. 2A, the optical sensor 116 is positioned rearwardly (i.e. further away from the retinal surface) from the tip or distal end of the tubular structure, and therefore rearward of the opening 112 which is formed in a lateral or side wall of the tubular structure. The cutting opening 112 is located slightly rearward of the tip of the tubular structure. As can be seen from FIG. 2A, the optical sensor 116 therefore produces an optical signal 118 that extends across the opening 112 and towards the retinal surface. In this fashion, the optical signal 118 is positioned so as to interact with any material which may approach the opening 112.

With additional reference to FIG. 2B, it should be noted that in certain embodiments, the optical sensor 116 may produce multiple light beams 118. For example, the optical sensor 116 may include multiple optical fiber-based broadband light sources affixed to the tubular structure of the vitrector 110, which may be adjacent to one another, or disposed in any suitable fashion. In one embodiment, the optical sensor 116 produces three light beams 118: one superimposed on one side of the opening 112, a second superimposed on a radially-opposite side of the opening 112, and a third located therebetween. Other approaches are also considered. For instance, in the embodiment illustrated in FIG. 2B, the optical sensor 116 produces multiple light beams 118 which are distributed across the opening 112.

It should be noted that the positioning of the optical sensor 116 on the outer surface of the tubular structure of the vitrector 110 may improve the ability of the optical sensor 116 to detect material approaching the opening 112. In addition, the presence of the guillotine 114 which moves within the tubular structure of the vitrector 110 may be impeded by locating elements within the tubular structure. As a result, the positioning of the optical sensor 116 on the outer surface of the tubular structure of the vitrector 110 may ensure that the guillotine 114 may operate substantially unimpeded.

The vitrectomy machine 240 is coupled to the vitrector 110, and provides the vitrector 110 with suction force, pneumatic pressure, electrical power, and the like. In certain embodiments a fragmatome may also be used, in conjunction with the vitrector 110, to deliver acoustic energy (e.g. ultrasounds). In addition, the vitrectomy machine may provide the vitrector 110 with instructions to control the operation of the vitrector 110. Example instructions may include instructions relating to the speed and frequency of movement of the guillotine, the degree to which the opening 112 should be open or closed, or the like. In addition, the vitrectomy machine 240 may be configured for issuing emergency instructions, for instance to cause an emergency-related stop of the movement of the guillotine, an emergency-related close of the opening 112, or the like.

The optical sensor 116 is coupled to the control system 120, described earlier in relation to FIG. 1. It should be noted that although illustrated here as being separate and external to the vitrectomy machine 240, in some other embodiments the control system 120 may form part and be integral to the vitrectomy machine 240. For example, existing vitrectors 110 and vitrectomy machines 240 may be retrofitted to incorporate the optical sensor 116, which may be affixed to the outside of the tubular structure of the vitrector 110, and to incorporate the related control system 120. Newly-constructed vitrectors 110 and associated vitrectomy machines 240 may be built with the optical sensor 116 already integrated within the vitrector 110, and with the control system 120 being implemented by the vitrectomy machine 240.

The control system 120 includes an optical coherence tomography (OCT) unit 210, a detection algorithm 220, and a feedback mechanism 230. The control system 120 is communicatively coupled to the optical sensor 116 for receiving information therefrom relating to the behaviour of the optical signal 118. In addition, the control system 120 is communicatively coupled to the vitrectomy machine 240 for providing it with information relating to analysis of the behaviour of the optical signal 118 performed by the control system 120.

The OCT unit 210 receives information from the optical sensor 116, for instance during a vitrectomy procedure. In some embodiments, the optical sensor 116 provides light scattering information which is indicative of a degree to which the optical signal 118 is scattered by the medium or material located in front of the optical sensor 116. In some other embodiments, the optical sensor 116 may provide different types of optical feedback relating to the way in which the optical signal 118 interacts with, or is interfered with, by the medium or material located in front of the optical sensor 116. The information may be provided as an analog signal, for instance a signal based on light which is interpreted by the OCT unit 210. For example, the OCT unit 210 may incorporate a spectrometer or similar device for processing an optical signal received from the optical sensor 116. The OCT unit 210 receives the optical signal and processes it, for instance by performing an OCT procedure using the optical signal. This may include comparing the received optical signal to a reference optical signal, to determine the behaviour of the optical signal 118 in the medium or material located in front of the optical sensor 116. The OCT unit 210 generates imaging data, which may include one or more amplitude scans (referred to as A-scans), and which is made available for use by the detection algorithm. For example, the OCT unit 210 may cause the A-scans or other imaging data to be stored within a memory or other data store within the control system 120.

The imaging data produced by the OCT unit 210 is used by the detection algorithm 220 to perform one or more detection steps. The detection algorithm 220 may be implemented by the control system 120 to identify the medium or material located in front of the optical sensor 116, and thus in proximity to the opening 112 of the vitrector 110. In some embodiments, the detection algorithm 220 is configured for detecting whether the material located in proximity to the opening 112 is the aqueous/vitreous humour within the eyeball 102, or the retina 104. For example, the detection algorithm 220 is configured for detecting whether or not the retina 104 is about to, or is in the process of, entering the opening 112 to be cut by the guillotine 114 and/or aspirated by the vitrector 110. In another example, the detection algorithm 220 is configured for determining properties of the aqueous/vitreous humour within the eyeball 102, or the retina 104, such as whether the retina 104 is moving within the eyeball 102, how the retina 104 is disposed within the eyeball 102, or the like. The detection algorithm may be developed in any suitable fashion, for instance based on testing performed using ex vivo eyeballs.

In some embodiments, the OCT unit 210 and/or the detection algorithm may determine various other information based on the signals obtained from the optical sensor 116. For instance, the OCT unit 210 obtains a refractivity index, a transmissivity index, or some other value which indicates the way in which the optical signal 118 interacts with, or is interfered with, by the medium or material located in front of the optical sensor 116.

The detection algorithm 220 may produce various information based on the identification and/or detection performed. In some embodiments, the detection algorithm 220 determines a medical risk for the patient on which the vitrectomy procedure is being performed. The medical risk may be low or null when the detection algorithm 220 identifies the presence of the aqueous and/or vitreous humour, or other transparent, semi-transparent, or translucent fluids within the eyeball 102 in proximity to the opening 112. The medical risk may be high when the detection algorithm 220 identifies the presence of the retina 104 in proximity to the opening 112. A particular medical risk for the patient may be detect when the retina 104 is determined to be mobile or moving, or responding in a particular way to the action of the vitrector 110. Other types of medical risks may also be identified.

In some other embodiments, the detection algorithm determines different information. For example, the detection algorithm 220 identifies the nature of the material in proximity to the opening 112. In another example, the detection algorithm 220 identifies a distance to the retina 104 from the optical sensor 116. In a further example, the detection algorithm 220 determines a degree of mobility of the retina 104, a degree of traction of the vitreous humour within the eyeball 102, or the like. Still other determinations may be performed by the detection algorithm 220.

When the detection algorithm 220 detects a particular medical risk, a medical risk above a particular threshold, or any other suitable information, the detection algorithm 220 may issue a signal to the feedback mechanism 230. The feedback mechanism 230 is coupled to the vitrectomy machine 240 to effect a change in the operation of the vitrector 110 in response to the information determined by the detection algorithm 220. In some embodiments, such as the one illustrated in FIGS. 2A-B, the feedback mechanism 230 provides a command to the vitrectomy machine 240 which instructs the vitrectomy machine 240 to change the operation of the vitrector 110. This may include instructing the vitrectomy machine 240 to cease providing suction force to the vitrector 110, to cause the opening 112 of the vitrector 110 to be shut, to slow or stop the movement of the guillotine 114, or the like.

In embodiments in which the control system 120 is separate and external to the vitrectomy machine 240, including embodiments in which an existing vitrectomy machine 240 is retrofitted to operate with the control system and the optical sensor 116, the feedback mechanism 230 may be configured for interfacing with an input used by the surgeon to operate the vitrector 110. For instance, the vitrector 110 may be operated by a foot pedal or similar input device. In such instances, the feedback mechanism 230 may be configured for interacting with the foot pedal. This may include issuing a signal to a controller associated with the foot pedal to override the input provided by the surgeon, or commanding a mechanical device to interfere or override the normal operation of the foot pedal. In embodiments in which the control system 120 forms part of and is integral to the vitrectomy machine 240, the feedback mechanism 230 provides commands to the vitrector 110 itself, or substantially directly controls the operation of the vitrector 110. This may include reducing or cutting suction force to the vitrector 110, shutting the opening 112 of the vitrector 110, slowing or stopping the movement of the guillotine 114, or the like.

In this fashion, the information obtained by the control system 120 from the optical sensor 116 may be used to assess or detect a medical risk for a patient undergoing a vitrectomy procedure. For instance, the control system 120 may identify a high degree of mobility of the retina 104, or that the retina 104 is about to enter the opening 112 of the vitrector 110. The control system 120 may then alter the operation of the vitrector 110 to avoid or minimize the risk of damage to the retina 104 or other portions of the eyeball 102 of the patient. In certain embodiments, the electronic nature of the control system 120 and the optical sensor 116 allow for response times exceeding those of an average surgeon, thereby reducing the risk of damage to the eyeball 102 of the patient. In some cases, a sufficiently rapid response time may result in avoiding or preventing damage to the retina 104 during vitrectomy procedures.

The attachment of an optical sensor 116 to the tubular structure of the vitrector 110 may increase its overall thickness (i.e. to increase the dimension of its transverse cross-section). A thicker vitrector may not be as readily compatible with standardized trocars used in clinical practice. Trocars are fixed on the eyeball to enable tool insertion and manipulation, and are usually provided together with the vitrector and match its size. The smaller the trocar the smaller the incision at the eyeball, thereby reducing the invasiveness of required surgical operations. It is therefore desirable that a vitrector 110, modified with the addition of the optical light sensor 116, fits inside a standard sized trocar that would be used with an unmodified vitrector.

In those embodiments wherein the optical sensor 116 comprises multiple optical fiber-based light sources (the “fiber-based optical sensor”), an alternative method to attach the optical sensor 116 to the vitrector may be considered to reduce the overall thickness of the vitrector 110 and make it compatible with standard trocars. The method, described in greater detail herein below, involves creating a lengthwise groove along the surface of the vitrector 110 in which a miniaturized fiber-based optical sensor may be placed, the fiber-based optical sensor being thereby embedded within the tubular structure of the vitrector 110, leading to a correspondingly lower thickness (i.e. a smaller transverse cross-section) of the vitrector 110 compared to the unmodified vitrector 110.

With additional reference to FIG. 2C, an exemplary method for grooving a vitrector 110 is illustrated. A laser source, for instance a nanosecond laser source 250, may be used to make a groove 252 on the surface of the vitrector 110. The laser source may be stationary or it may be in motion. In one embodiment, the vitrector 110 may be attached to a motorized stage 260 allowing translation of the vitrector 110 in respect to a focused laser beam 254 emitted by the nanosecond laser source 250, while the nanosecond laser source 250 is stationary with respect to the vitrector 110. The laser beam 254 may be configured using some pre-defined settings in terms of pulse energy, repetition rate, pulse direction, beam size at the focal point, and/or wavelength. For instance, the laser beam 254 may be configured with a pulse energy of 3.5 mJ, a pulse repetition rate of 30 Hz, a pulse direction of 6 ns, a beam size at the focal point of 65 μm, and a wavelength of 532 nm.

In one embodiment, a scanning approach may be used to make the groove 252 on the surface of the vitrector 110, wherein, as the vitrector 110 is displaced with a back-and-forth motion 258 along its longitudinal axis 270 by the motorized stage 260, the nanosecond laser source 250 scans the surface of the vitrector 110 and makes at least one set of line scans, for instance the set of line scans 256, each set of line scans consisting of a plurality of line scan segments, and wherein each set of line scans is separated from the other set(s) of line scans by a pre-specified distance. In one embodiment, 3 sets of 50 line scans of the vitrector 110 may be created at 0.5 mm/s, and the distance between the sets of line scans may be 50 μm. The resulting groove 252 may be created along the longitudinal axis 270 of the vitrector 110, and the groove 252 may be aligned with the opening 112 of the vitrector 110. However, it should be understood that the grooving process may not be limited to the nanosecond laser source 250 and/or scanning conditions outlined above. Any known laser micromachining process may be used, including the use of continuous wave, picosecond, nanosecond, or femtosecond lasers. Furthermore, laser beam scanning may be used instead of vitrector displacement using the motorized stage 260.

FIG. 2D illustrates a transverse cross-sectional view of vitrector 110. The groove 252 may be created using the grooving process outlined above. A fiber-based optical sensor 280 may be miniaturized using a known approach, for instance using hydrofluoric acid (HA) etching to reduce the diameter of fiber-based optical sensor 280. The fiber-based optical sensor 280 may be, for example, single mode fiber or multimode fiber. A certain length of a long coreless fiber of fiber-based optical sensor 280, for instance a 100 μm length, may be spliced to protect the latter's core from the hydrofluoric acid, and then etched for some period of time in hydrofluoric acid, e.g. for 27 minutes. This process has been found to reduce the diameter of fiber-based optical sensor 280 substantially, for example, by up to 60%, e.g. from 125 μm to 50 μm.

With continued reference to FIG. 2D, the fiber-based optical sensor 280, miniaturized using an approach outlined above, may then be positioned along the groove 252 of the vitrector 110. In some embodiments, the fiber-based optical sensor 280 is glued along the groove 252 of the vitrector 110. In one embodiment, an assembly prepared using 25+ G Alcon vitrector may fit inside a 25 G trocar, where the excess thickness of the assembly compared to the unmodified vitrector is 25 μm.

It should be recognized that the process outlined above may apply to vitrectors of any size, i.e. 23 G, 25 G, and 27 G. Longer etching times may further reduce the thickness of the fiber-based optical sensor 280. The current miniaturization limit may be as low as 10 μm.

In some embodiments as illustrated in FIG. 2E with additional reference to FIGS. 2B-D, the optical detection system of vitrector 110 which is operable to detect the presence of the retina 104 in proximity to the cutting member of the vitrector 110 may include a fiber-based optical sensor 280 attached to the vitrector 110 as described herein above. A reflective element 294 may be attached to the distal end of vitrector 110 opposite to the opening 112 of vitrector 110. A laser source 284 emitting a laser beam 286 may be coupled to the fiber-based optical sensor 280 using laser coupling optics 282 which enable forward propagation of laser beam 286 into fiber-based optical sensor 280 but prevent back-propagation of the reflected laser beam 286 into the laser source 284. Once emitted, the laser beam 286 propagates through the laser coupling optics 282 and the fiber-based optical sensor 280, across the opening 112 of vitrector 110, through the retina 104 located in proximity to the cutting member of the vitrector 110 (if applicable), and is incident upon the reflective element 294, whereupon it undergoes reflection and back-propagates along substantially the same trajectory. The presence of material in proximity to the cutting member of the vitrector 110, for instance the retina 104, may alter the magnitude of the reflected beam 286. The laser coupling optics 282 may further enable propagation of the back-reflected laser beam 286 into a back reflection sensor 290 to monitor the power of the back reflected laser beam 286. A reference sensor 288 may be provided to monitor the power of the laser source 284. A detection algorithm 292 may trigger a shutdown of the vitrectomy machine 240 if sufficient variation of back reflected laser power is detected. It should be understood that other possibilities than those described above may apply depending on the application. For example, the wavelength of the laser source 284 may be selected in a different spectrum, for example in the UV, visible, or IR spectra. The fiber-based optical sensor 280 may comprise different fiber optics technologies, for example single mode or multimode fiber, and may include a focusing design. The laser source 284 may be continuous wave or pulsed wave.

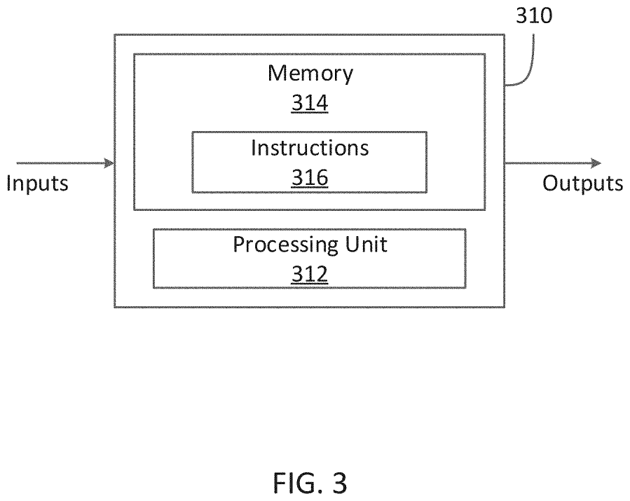

Referring now to FIG. 3, part or all of the embodiments of the devices, systems and methods described herein may be implemented in a combination of both hardware and software. These embodiments may be implemented on programmable computers, each computer including at least one processor, a data storage device or system (including volatile memory or non-volatile memory or other data storage elements or a combination thereof), and at least one communication interface. Throughout the present disclosure, numerous references may be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

For example, part or all of the control system 120 may be implemented by a computing device 310, comprising a processing unit 312 and a memory 314 which has stored thereon computer-executable instructions 316. It should be noted that the vitrectomy machine may also incorporate one or more computing devices 310, which may be used to implement the control system 120 in certain embodiments. For simplicity only one computing device 310 is shown but system may include more computing devices 310. The computing devices 310 may be the same or different types of devices.

The processing unit 312 may comprise any suitable devices configured to implement the functionality ascribed to the monitoring and control system 120 such that instructions 316, when executed by the computing device 310 or other programmable apparatus, may cause implementation of some or all of the functionality ascribed to the monitoring and control system 120 described herein. The processing unit 312 may comprise, for example, any type of microprocessor or microcontroller, a digital signal processing (DSP) processor, a central processing unit (CPU), an integrated circuit, a field programmable gate array (FPGA), a reconfigurable processor, other suitably programmed or programmable logic circuits, or any combination thereof.

The memory 314 may comprise any suitable known or other machine-readable storage medium. The memory 314 may comprise non-transitory computer readable storage medium, for example, but not limited to, an electronic, magnetic, optical, electromagnetic, infrared, or semiconductor system, apparatus, or device, or any suitable combination of the foregoing. The memory 314 may include a suitable combination of any type of computer memory that is located either internally or externally to device, for example random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Memory 314 may comprise any storage means (e.g., devices) suitable for retrievably storing the machine-readable instructions 316 executable by processing unit 312.

The instructions 316 are applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

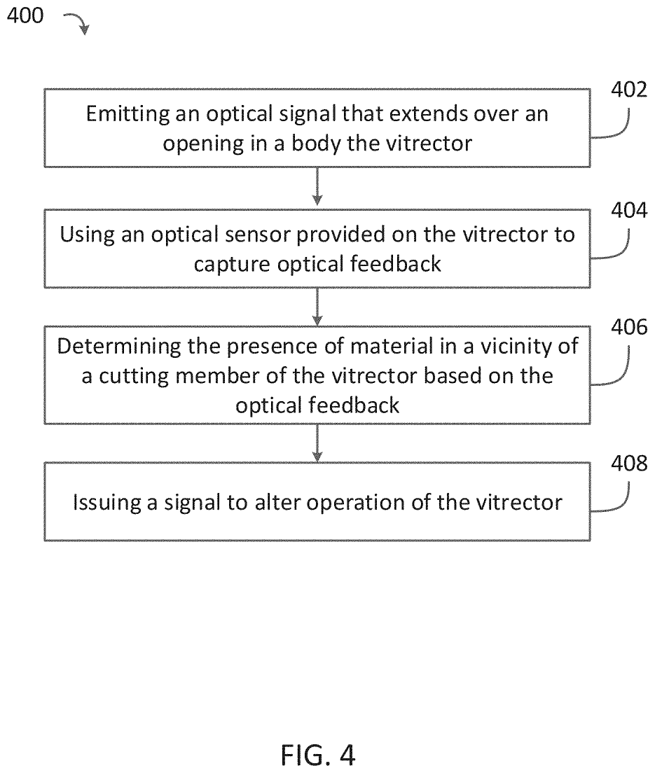

With reference to FIG. 4, there is illustrated a method 400 for controlling a vitrector, for instance the vitrector 110. At step 402, the method 400 comprises emitting an optical signal that extends over an opening in a body of the vitrector 110, for instance the opening 112. In one embodiment, the optical feedback may comprise light scattering information which may include one or more analog signals, one or more digital signals, and may include any information indicative of a degree to which the optical signal 118 is scattered by the medium or material located in front of the optical sensor 116. In another embodiment, the optical feedback may comprise a coherent light signal, for instance the back-reflected laser beam 286.

At step 404, the method 400 comprises using an optical sensor provided on the vitrector 110, for instance optical sensor 116 or fiber-based optical sensor 280, to capture optical feedback. The optical feedback may be produced when the optical sensor interacts with material proximate to an opening of vitrector 110. In some embodiments, the optical feedback may comprise light scattering information, and imaging data may be generated by performing OCT based on the light scattering information. In some embodiments, one or more spectrometers may be used to produce the imaging data, which may include one or more A-scans. In some other embodiments, other imaging data may be produced, as appropriate.

At step 406, the method 400 comprises determining the presence of the material in a vicinity of the cutting member of the vitrector 110, for instance the guillotine 114, based on the optical feedback. The material may relate to the retina 104 of the patient, or to another portion of the eyeball 102 of the patient. For example, the material may involve the retina 104 approaching or being about to enter the opening 112 of the vitrector 110. In another example, the material may involve the retina 104 exhibiting a degree of mobility, or a likelihood of a degree of mobility above a predetermined threshold. Other types of material may also be detected, depending on the application.

At step 408, the method 400 comprises issuing a signal to alter the operation of the vitrector 110 responsive to determining the presence of material in the vicinity of the cutting member of the vitrector 110, for instance the guillotine 114. The signal may result in a change in the amount of suction force provided to the vitrector 110, in a change to the movement or behaviour of the guillotine 114, for instance halting or otherwise ending the movement of the guillotine 114, a closing of the opening 112, or the like. The signal may be issued to the vitrector 110 itself, or to a system which controls the vitrector 110.

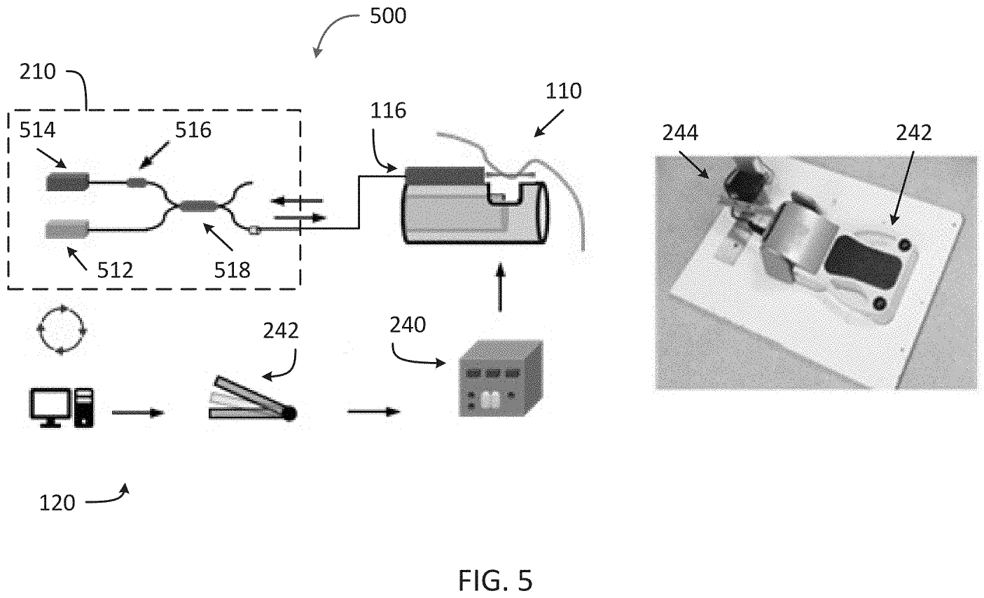

With reference to FIG. 5, an exemplary embodiment of a vitrectomy system 500 is illustrated. In the exemplary vitrectomy system 500, the optical sensor 116 is provided with light via a broadband light source 514. In some embodiments, an attenuator 516 is provided in the path between the broadband light source 514 and the optical sensor 116 to alter the amplitude, or other characteristics, of the light provided by the broadband light source 514. A spectrometer 512 is coupled to the optical sensor 116 and the broadband light source 514 via a coupler 518, and receives a signal from the optical sensor 116. In some embodiments, the OCT unit 210 includes the spectrometer 512, the broadband light source 514, the attenuator 516 and the coupler 518, and operates using the common-path configuration illustrated in FIG. 5.

In one embodiment, the OCT unit 210 operates at a central wavelength of 840 nm and a bandwidth of 50 nm and provides A-scans at a frequency of 100 Hz. In another embodiment, the OCT unit 210 provides A-scans at a frequency of 500 Hz. The broadband light source 514 may be implemented by a SLED light source (having a central wavelength of 880 nm and a bandwidth of 70 nm) powered by a compact driver board. The spectrometer 512 may be any suitable spectrometer, and the coupler 518 may be a 50:50 fiber coupler. The attenuator 516 may be a variable optical attenuator of any suitable type.

The OCT unit 210 may be controlled by various elements of the control system 120. For example, a LabVIEW-based program may be used. The A-scans may be acquired by the control system 120 using evenly spaced wavelength data which are converted into even k-space data with linear interpolation. The raw interferogram data is filtered with a high-pass filter to remove a DC component, and then with a low-pass filter to remove high frequency noise portions of the signal. A fast-Fourier transform (FFT) is applied to the filtered interferogram to acquire the A-scans. It should be noted that some approaches make use of an imaging module which scans the optical signal 118 as it exits the optical sensor 116, and in some other approaches, no imaging module is employed. The detection algorithm 230 of the control system 120 may be used to interrogate the presence of the retina 104 in front of the opening 112 of the vitrector 110 by processing the A-scans substantially in real-time.

During certain vitrectomy procedures, a surgeon controls the amount of vacuum produced by the vitrector by using a foot pedal, illustrated at 242, connected to the vitrectomy machine. Typically, the cutting rate of the cutting member of the vitrector may be kept at a given fixed speed (e.g. a maximum cutting rate, for example, which may be proportional to the vacuum mode). Both the vacuum pressure applied and the cutting rate of the vitrector may also be controlled, individually and/or simultaneously, in a different operational mode of the vitrectomy machine. Acutating the pedal may thus initiates the negative pressure (vacuum) function of the vitrector 110 and the movement of the cutting member (i.e., reciprocal movement of the guillotine 114) within the vitrector is separately actuated to set a cutting rate of the vitrector. During PPV surgery, the surgeon must quickly release the foot pedal 242 when a dangerous situation is observed, for instance the retina 104 approaching the guillotine 114, such as to immediately de-activate the vacuum. In the exemplary embodiment of FIG. 5, a robotic arm 244 is operated by the control system 120 to move the foot pedal 242, for instance by way of a servo motor, which form part of the feedback mechanism 230. When the control system 120 detects a medical risk for the patient, the feedback mechanism 230 causes the foot pedal 242 to release, via the robotic arm 244, thereby reducing or minimizing the risk of damage to the retina 104.

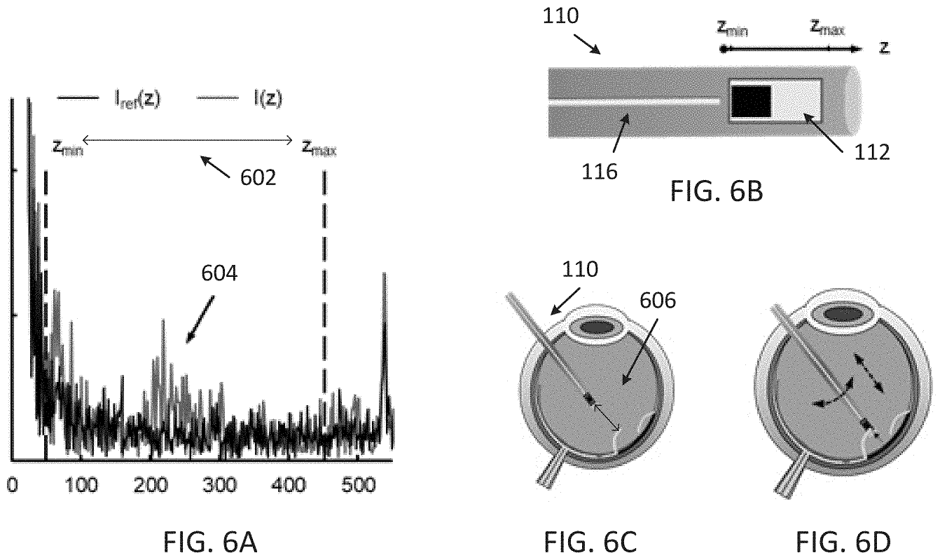

With reference to FIGS. 6A-D, an example approach for developing the detection algorithm 220 is presented. Using an in vivo experimentation scenario, the vitrector 110 is positioned at a certain distance from the retina, illustrated at 606 in FIG. 6C. For example, the distance 606 may be greater than a few millimeters, for instance more than 5 mm. The vitrector 110 is then used to acquire one or more reference A-scans (noted as I_{ref}(z)). As illustrated in FIG. 6A, an interrogating window 602 is selected, illustrated here as zₘᵢₙ to zₘₐₓ, which corresponds to the length of the opening 112 of the vitrector 100, which may be approximately 400 micrometers (μm). In one embodiment, the value of zₘᵢₙ is varied from 100 μm to 200 μm, depending on the offset of the optical sensor 116 to the opening 112, and zₘₐₓ is set to zₘᵢₙ+400 μm . Values of the reference A-scans I_{ref}(z) obtained at the distance 606 are used as a comparison point for intraoperative A-scans obtained substantially in real-time during a vitrectomy procedure, as illustrated in FIG. 6D (and noted as I(z)). In particular, A-scan values I(z) for the amplitude of the optical signal 118 detected by the optical sensor 116 and the OCT unit 210 obtained during vitrectomy procedures may be compared to the reference A-scans. Peaks in the A-scan values I(z), such as those illustrated at 604 in FIG. 6A, may be indicative of the presence of the retina 104, rather than the vitreous humour within the eyeball 102.

In one embodiment, the detection algorithm 220 is configured for detecting the presence of the retina 104, and thus commanding the feedback mechanism 230 to activate, when at least 1-2% of the A-scan values I(z) within the interrogated window 602 met the condition

I(z)>S·I_{ref}(z)

where S is a predetermined parameter, and z is the distance between the optical sensor 116 and a sensed object ahead of the optical sensor 116. In this embodiment, the detection algorithm 220 is capable of identifying the presence of the retina 104 when it covers approximately 1-2% of the opening 112 of the vitrector 110.

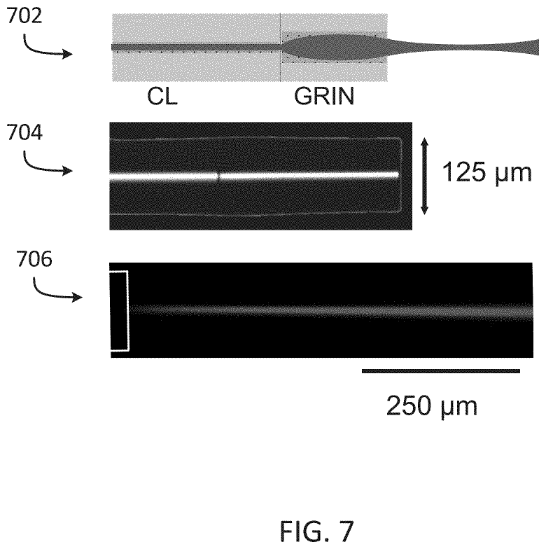

With reference to FIG. 7, example light outputs of example broadband light sources are illustrated. In an exemplary embodiment, the optical sensor 116 and portions of the OCT unit 210 (for instance, the broadband light source 514) may include various optical fibers, for instance commercially-available optical fibers which may have been spliced of cleaved using any suitable equipment. The initial thickness of the optical fibers, prior to be coupled to the vitrector 110, may be approximately 125 μm. The optical fibers may be cut to focus the optical signal 118 exiting the optical sensor 116 at a particular distance, for instance at approximately 260 μm from a distal tip thereof once immersed in the aqueous/vitreous humour. The design of the optical sensor 116, including any cuts or shapes, may be determined using a theoretical model. In one particular embodiment, a 320 μm GRIN fiber component is used to provide the focusing aspect. The optical sensor 116 is spliced to a 2-meter long SM fiber FC/APC patch cable, which is illustrated schematically at 702 and using optical microscopy at 704. A visualization of the optical signal 118 exiting the optical sensor 116 into the aqueous/vitreous humour is illustrated schematically at 706.

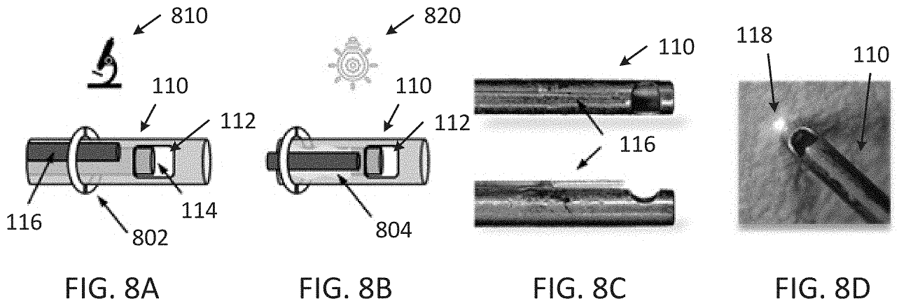

With reference to FIGS. 8A-D, an example assembly process for the vitrector 110 is illustrated. The degree of precision of the optical sensor 116 is dependent on how the optical sensor 116 is affixed and aligned on the tubular structure of the vitrector 110. In some embodiments, the distal end of the optical sensor 116 is positioned to point in front of the opening 112, with a gap of approximately 100 μm to 200 μm between the distal end of the optical sensor 116 and the proximate end of the opening 112. To affix the optical sensor 116, the tubular structure of the vitrector 110 is mounted to a first translation stage, and the optical sensor 116 is affixed to a second translation stage. The two stages are then aligned under a microscope 810 using plastic rings 802 to facilitate the alignment. An adhesive 804, for instance an adhesive designed for medical products, is used to affix the optical sensor 116 to the tubular structure of the vitrector 110. FIGS. 8A and 8B schematically illustrate the assembly process, and FIG. 8C illustrates top and side views of an example vitrector 110. An example of the optical signal 118 is illustrated in FIG. 8D.

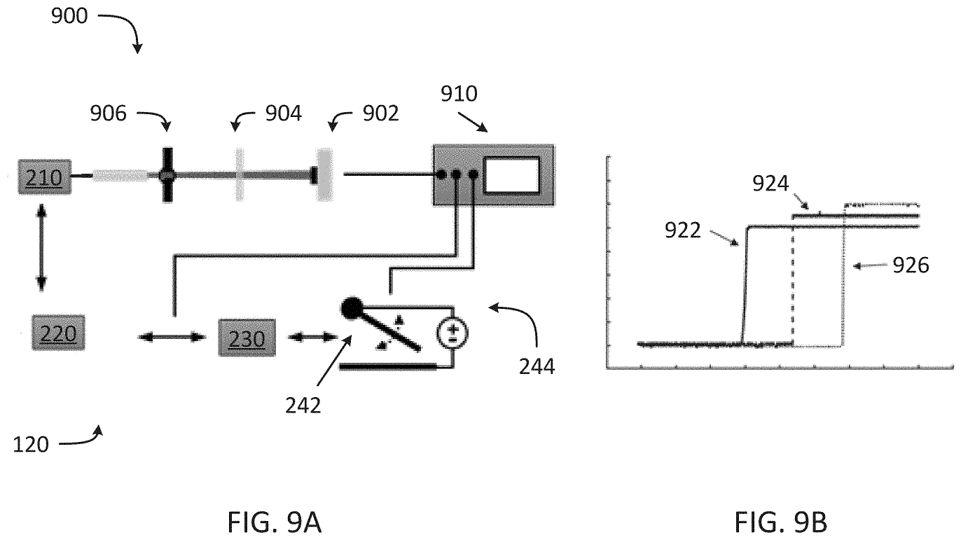

With reference to FIGS. 9A-B, an example testing setup 900 for the feedback mechanism 230 is illustrated. The testing setup 900 includes a signal processing device 910, as well as a photodiode 902, a cover slip 904, and a shutter 906. The testing setup 900 is used to measure the response time of the vitrector 110 in different operating scenarios. An example testing scenario involves triggering the detection algorithm of the control system 120. A cover slip 904 is used to mimic the presence of the retina 104, and a photodiode 902 is used to provide the reference time (delay <2 ns) that the optical signal 118 hits the cover slip 904. As a result, when the shutter 906 is actuated, the OCT unit 210 receives a signal which is representative of what the optical sensor 116 would provide the OCT 210 when the retina 104 is proximate to the opening 112 of the vitrector 110.

The OCT unit 210 generates A-scans, which are provided to the other elements of the control system 210, including for instance the detection algorithm 220. When the shutter is actuated, one or more A-scans are generated by the OCT unit which mimic the retina 104 being proximate to the opening 112, acting as a triggering event for the detection algorithm 220. The response of both the detection algorithm 220 and the feedback mechanism 230 actuating the robotic arm 244 are timed by the signal processing device 910. As illustrated in FIG. 9B, the photodiode trigger signal is illustrated as curve 922. The response provided by the detection algorithm is shown as curve 924, and the actuation of the robotic arm 244 is shown as curve 926.

In some exemplary embodiments, the delay between the photodiode trigger 922 and the response of the detection algorithm 924 is on the order of 0.01 seconds, and the delay until the actuation of the robotic arm 244 is less than 0.03 seconds. This response time may serve to reduce the number of non-desirable cuts performed by the vitrector 110. For example, if the guillotine 114 of the vitrector 110 operates at a rate of 5000 cuts per minute, the robotic arm 244 actuating the foot pedal 242 may result in approximately 2 to 3 undesirable cuts; the response time of a typical surgeon may result in 30 or more undesirable cuts. Additionally, the amount of movement performed by the vitrector 110 as part of the halting of the guillotine 114 may be reduced vis-à-vis the typical response of the surgeon. It should be noted that in this example, the time associated with the generation of the A-scan and detection of the retina 104 represent a small fraction of the total response time, with the bulk residing in the response time of the motor controlling the robotic arm 244. Quicker response times may be obtained by using electronic switches or more quickly-responding servo motors. Additionally, embodiments in which the control system 120 is integrated within the vitrectomy machine 240 may be configured to more directly control the operation of the vitrector 110, further reducing the response time.

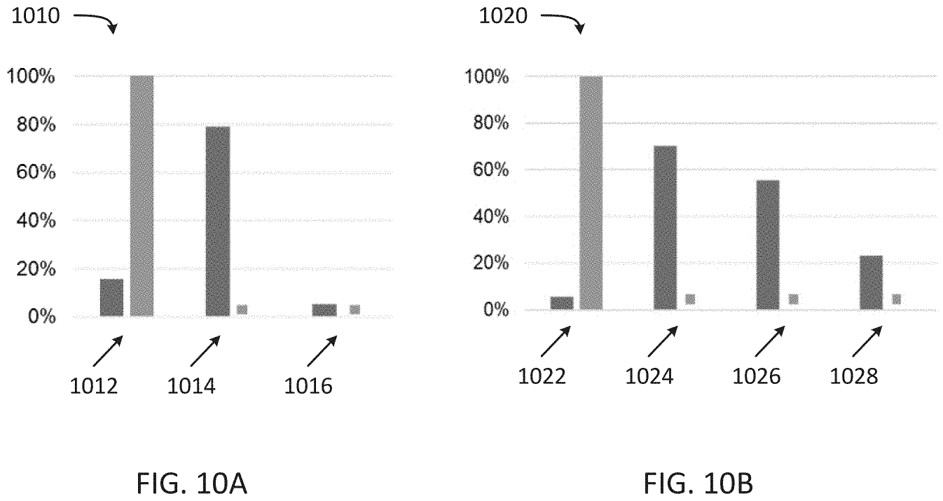

With reference to FIGS. 10A-B, results of both ex vivo and in vivo testing of the vitrectomy system 100 are illustrated. In some cases, the experimentation involves enucleated porcine eyes in which vitrectomy maneuvers were performed by a vitreoretinal surgeon using the vitrector 110 with or without activating the operation of the control system 120. The surgeon performed multiple aggressive approaches with an intention to injure the retina 104, which involved operating close to both attached retina 104 and detached/torn retina 104, and the outcomes of each approach were evaluated.

In FIG. 10A, three evaluation scores were used, and are displayed in the chart 1010: retinal injury (i.e., no cutter de-activation at all, “retinal bite”) at 1012; retinal injury prevention (i.e., on-time de-activation of the cutter) at 1014; and early stop (i.e., irrelevant de-activation of the “cutter” far from the retina) at 1016. The chart 1010 illustrates values in pairs, with the left bars relating to experiments using the vitrectomy system 100 with the operation of the control system 120, and the right bars relating to experiments performed with a standard vitrectomy system. As illustrated at 1012, use of the vitrectomy system 100 results in reduced retinal injury vis-à-vis a standard vitrectomy system. And while the standard vitrectomy system does not prevent any retinal injury (at 1014) and does not result in any early stops of the vitrector (at 1016), the vitrectomy system 100 does prevent retinal injury (at 1014) and additionally does not produce any early stops of the vitrector (at 1016).

In FIG. 10B, four evaluation scores were used, and are displayed in the chart 1020: retinal injury at 1022; retinal injury prevention or mitigation at 1024; retinal injury prevention alone at 1026; and early stop at 1028. The chart 1020 illustrates values in pairs, with the left bars relating to experiments using the vitrectomy system 100 with the operation of the control system 120, and the right bars relating to experiments performed with a standard vitrectomy system. As illustrated at 1022, use of the vitrectomy system 100 results in reduced retinal injury vis-à-vis a standard vitrectomy system. The vitrectomy system 100 prevents or mitigates retinal injury (at 1024 and 1026) at higher rates than the standard vitrectomy system, and early stopping of the vitrector 110 is at a relatively low rate (at 1028).

The present disclosure provides many example embodiments. Although each embodiment represents a single combination of inventive elements, other examples may include all possible combinations of the disclosed elements. Thus if one embodiment comprises elements A, B, and C, and a second embodiment comprises elements B and D, other remaining combinations of A, B, C, or D, may also be used. In addition, the term “connected” or “coupled to” may include both direct coupling (in which two elements that are coupled to each other contact each other) and indirect coupling (in which at least one additional element is located between the two elements).

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements. The embodiments described herein are directed to electronic machines and methods implemented by electronic machines adapted for processing and transforming electromagnetic signals which represent various types of information. The embodiments described herein pervasively and integrally relate to machines, and their uses; and the embodiments described herein have no meaning or practical applicability outside their use with computer hardware, machines, and various hardware components. Substituting the physical hardware particularly configured to implement various acts for non-physical hardware, using mental steps for example, may substantially affect the way the embodiments work. Such computer hardware limitations are clearly essential elements of the embodiments described herein, and they cannot be omitted or substituted for mental means without having a material effect on the operation and structure of the embodiments described herein. The computer hardware is essential to implement the various embodiments described herein and is not merely used to perform steps expeditiously and in an efficient manner.

Although the embodiments have been described in detail, it should be understood that various changes, substitutions and alterations can be made herein without departing from the scope as defined by the appended claims.

Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps. As can be understood, the examples described above and illustrated are intended to be exemplary only.

What is claimed is:

1. A method for controlling a vitrector of a vitrectomy system, the method comprising:
emitting an optical signal over an opening in a body of the vitrector, the opening providing access to a cutting member within the body;
using an optical sensor provided on the vitrector to capture optical feedback produced when the optical signal interacts with material proximate to the opening;
determining the presence of the material in a vicinity of the cutting member of the vitrector based on the optical feedback; and
issuing a control signal to alter operation of the vitrector responsive to determining the presence of the material in the vicinity of the cutting member of the vitrector.

2. The method of claim 1, further comprising processing the optical feedback and issuing a detection result.

3. The method of claim 2, wherein the optical feedback comprises light scattering information, and wherein processing the light scattering information comprises generating imaging data by performing optical coherence tomography, and further wherein issuing the detection result comprises providing the imaging data to a detection algorithm.

4. The method of claim 3, wherein generating the imaging data by performing optical coherence tomography comprises generating at least one amplitude scan based on the light scattering information.

5. The method of claim 2, wherein the optical signal comprises a laser beam propagating in a first direction and the optical feedback comprises a back-reflection of the laser beam.

6. The method of claim 5, wherein processing the optical feedback comprises detecting a variation of a power of the laser beam and the back-reflection of the laser beam, and wherein issuing a detection result comprises providing the variation of power to a detection algorithm.

7. The method of claim 1, wherein the optical sensor is disposed on an outer surface of the body of the vitrector.

8. The method of claim 1, wherein the optical sensor comprises a fiber-based optical sensor positioned inside a groove formed in an outer surface of the body of the vitrector and extending longitudinally therealong.

9. The method of claim 1, wherein determining the presence of the material in the vicinity of the cutting member of the vitrector comprises determining whether a retina is approaching the opening of the vitrector.

10. The method of claim 1, wherein the material is a retina, and further wherein determining the presence of the material in the vicinity of the cutting member of the vitrector further comprises determining a presence of an aqueous/vitreous humour of the patient in the vicinity of the opening of the vitrector.

11. The method of claim 1, wherein issuing the control signal to alter the operation of the vitrector comprises commanding a stopping of movement of the cutting member of the vitrector.

12. The method of claim 1, wherein issuing the control signal to alter the operation of the vitrector comprises commanding a reduction in a suction force provided to the vitrector.

13. A vitrectomy system, comprising:
a vitrector including a tubular structure defining an opening near a distal end thereof for aspiration of material, and a cutting member located within the tubular structure and translating therein for cutting material which enters the tubular structure via the opening;
an optical sensor provided on the vitrector and aligned to emit an optical signal that extends over the opening and to capture optical feedback produced when the optical signal interacts with material proximate to the opening; and
a control system coupled to the optical sensor and the vitrector for:
receiving the optical feedback from the optical sensor;
determining the presence of the material in a vicinity of a cutting member of the vitrector based on the optical feedback; and
issuing a control signal to alter the operation of at least one of the cutting member and a suction force source coupled to the vitrector responsive to determining the presence of the material. The vitrectomy system of claim 13, wherein the optical sensor is disposed on an outer surface of the body of the vitrector.

14. The vitrectomy system of claim 13, wherein the optical sensor comprises a fiber-based optical sensor positioned inside a groove formed in an outer surface of the body of the vitrector, the groove extending longitudinally along the body of the vitrector.

15. The vitrectomy system of claim 13, wherein the control system coupled to the optical sensor comprises:
an optical coherence tomography (OCT) unit communicatively coupled to the optical sensor, the OCT unit configured for receiving optical feedback from the optical sensor, processing the optical feedback, and generating imaging data by performing optical coherence tomography;
a detection algorithm communicatively coupled to the OCT unit, the detection algorithm configured to detect the material proximate to the opening of the vitrector and issue a detection signal; and
a feedback mechanism communicatively coupled to the detection algorithm and to the vitrector, the feedback mechanism configured to effect a change in the operation of the vitrector in response to the detection signal.

16. The vitrectomy system of claim 15, wherein the control system further comprises:
a broadband light source providing light to the optical sensor;
an attenuator provided in a path between the broadband light source and the optical sensor; and
a spectrometer communicatively coupled to the optical sensor and to the broadband light source via a coupler, the spectrometer receiving a signal from the optical sensor.

17. The vitrectomy system of claim 16, wherein the OCT unit comprises the spectrometer, the broadband light source, the attenuator, and the coupler in a common-path configuration.

18. The vitrectomy system of claim 13, wherein the optical sensor is a fiber-based optical sensor, the vitrectomy system further comprises a reflective element provided on a distal end of the vitrector opposite to the opening, and wherein the control system coupled to the optical sensor further comprises:
a laser source configured for emitting a laser beam, the laser beam undergoing reflection on the reflective element;
a reference sensor configured for monitoring the power of the laser source;
a back-reflection sensor configured for monitoring the power of a back reflection of the laser beam;
laser coupling optics configured for coupling the laser source, the optical sensor, and the back-reflection sensor, the laser coupling optics enabling forward propagation of the laser beam into the fiber-based optical sensor but preventing back-propagation of the laser beam into the laser source; and
a detection algorithm configured for triggering a shutdown of the vitrectomy system is sufficient power variation of the back reflection of the laser beam is detected.

19. The vitrectomy system of claim 18, wherein the wavelength of the laser source is in the UV, visible, or IR spectra.

20. The vitrectomy system of claim 13, wherein the vitrectomy system further comprises a vitrectomy machine communicatively coupled to the control system and to the vitrector, the control system issuing commands to the vitrectomy machine for operating the vitrector.
